(19) United States
(12) Patent Application Publication
(10) Pub. No.: US 2017/0067863 A1
(43) Pub. Date: Mar. 9, 2017

(54) Portable spectroscopic analysis tool

(71) Applicant: Ranjith Kunnath Narayanan, Mason, OH (US)

(72) Inventor: Ranjith Kunnath Narayanan, Mason, OH (US)

(21) Appl. No.: 15/257,841

(22) Filed: Sep. 6, 2016

Related U.S. Application Data
(60) Provisional application No. 62/213,713, filed on Sep. 3, 2015.

(51) Int. Cl. G01N 30/72; G01N 33/02

(52) U.S. Cl. CPC G01N 30/7206; G01N 33/02

(57) ABSTRACT

A portable spectroscopy analysis tool tests food for herbicides, pesticides, growth hormones, carcinogens, and other materials. Due to the broad range of herbicides, pesticides, growth hormones, carcinogens, and other materials which may be subjects of concern for the user, and the immediate need for accurate test results based on a small sample, the tool preferably employs gas chromatography-mass spectrometry (GC-MS). Alternate embodiments, employing alternate methods of chromatography such as liquid chromatography (LC), are contemplated. The user dissolves a small shaving of a food sample into a volatile solvent, such as alcohol, and dips a test strip into the resulting solution. The strip is then inserted into an aperture which is provided on the top surface of the tool, and a visual display provides a chemical analysis of the sample within seconds.

### CROSS-REFERENCE TO RELATED APPLICATIONS

This Application claims the benefit of U.S. Provisional Application No. 62/213,713, filed Sep. 3, 2015, which is hereby incorporated by reference.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates generally to home health care equipment and in particular to a portable spectroscopy analysis tool. Across America, millions of consumers are demonstrating increased awareness and concern about the safety of their food and beverages. In particular, the presence of herbicides, pesticides, growth hormones, and carcinogens has become fairly commonplace due to large scale farming practices. When shopping for groceries or preparing to eat a meal in a restaurant, many consumers would appreciate having a tool to test various food items for the presence of such materials. A portable spectroscopy analysis tool, which may be used to quickly and accurately test food samples for the presence of such materials, would resolve this problem.

### SUMMARY OF THE INVENTION

Accordingly, the invention is directed to a portable spectroscopy analysis tool. The tool is similar in structure and function to the glucose meters used by diabetics for home blood testing, with the following modifications. Due to the broad range of herbicides, pesticides, growth hormones, carcinogens, and other materials which may be subjects of concern for the user, and the immediate need for accurate test results based on a small sample, the tool preferably employs gas chromatography-mass spectrometry (GC-MS). Alternate embodiments, employing alternate methods of chromatography such as liquid chromatography (LC), are contemplated. The user dissolves a small shaving of a food sample into a volatile solvent, such as alcohol, and dips a test strip into the resulting solution. The strip is then inserted into an aperture which is provided on the top surface of the tool, and a visual display provides a chemical analysis of the sample within seconds.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWINGS

The accompanying drawings are included to provide a further understanding of the invention and are incorporated into and constitute a part of the specification. They illustrate one embodiment of the invention and, together with the description, serve to explain the principles of the invention.

FIG. 1 is a front perspective view of the first exemplary embodiment, displaying the tool 10, the test strip 11, the visual display 13, and the activation button 14.

FIG. 2 is a top view of the first exemplary embodiment with the test strip removed, displaying the tool 10, and the aperture 12.

FIG. 3 is a right side view of the first exemplary embodiment with the test strip removed, displaying the tool 10, and the visual display 13.

FIG. 4 is a front view of the first exemplary embodiment, displaying the tool 10, the test strip 11, the visual display 13, the activation button 14, and the indicator light 15.

FIG. 5 is an interior view of the first exemplary embodiment, displaying the aperture 12, the left compartment 21, the right compartment 22, and the battery 40.

FIG. 6 is a system diagram of the means for testing the content of a sample. Specifically, a gas chromatograph combined with a mass spectrometer, displaying the carrier gas 22 which is connected to the column oven 23 of the gas chromatograph 30, the sample injector 24, the detector 25 and the mass spectrometer 26.

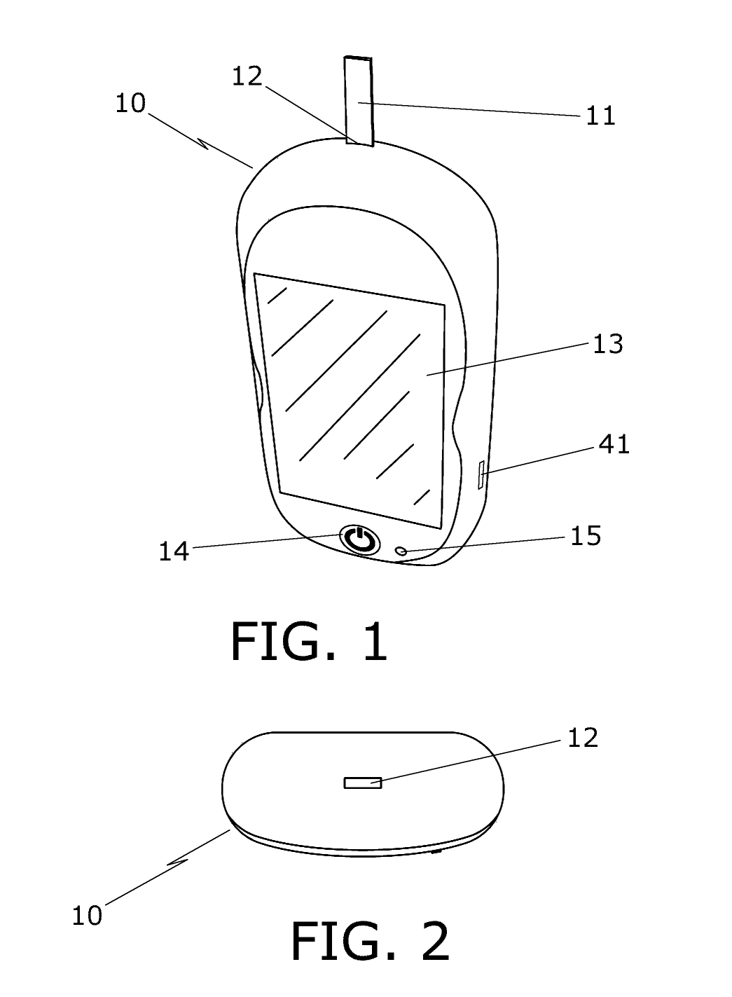

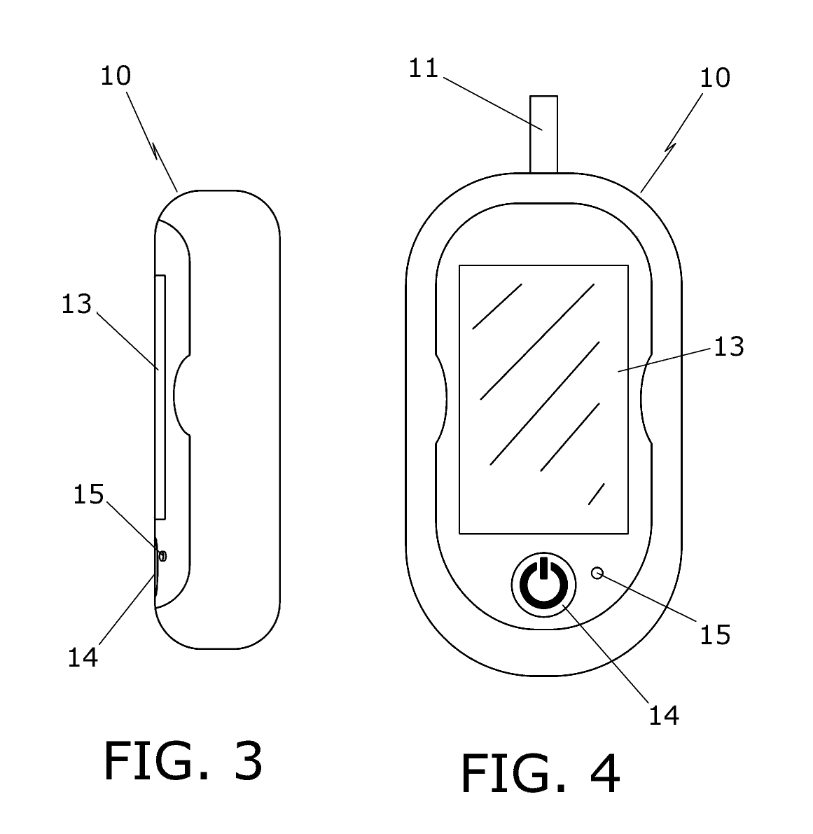

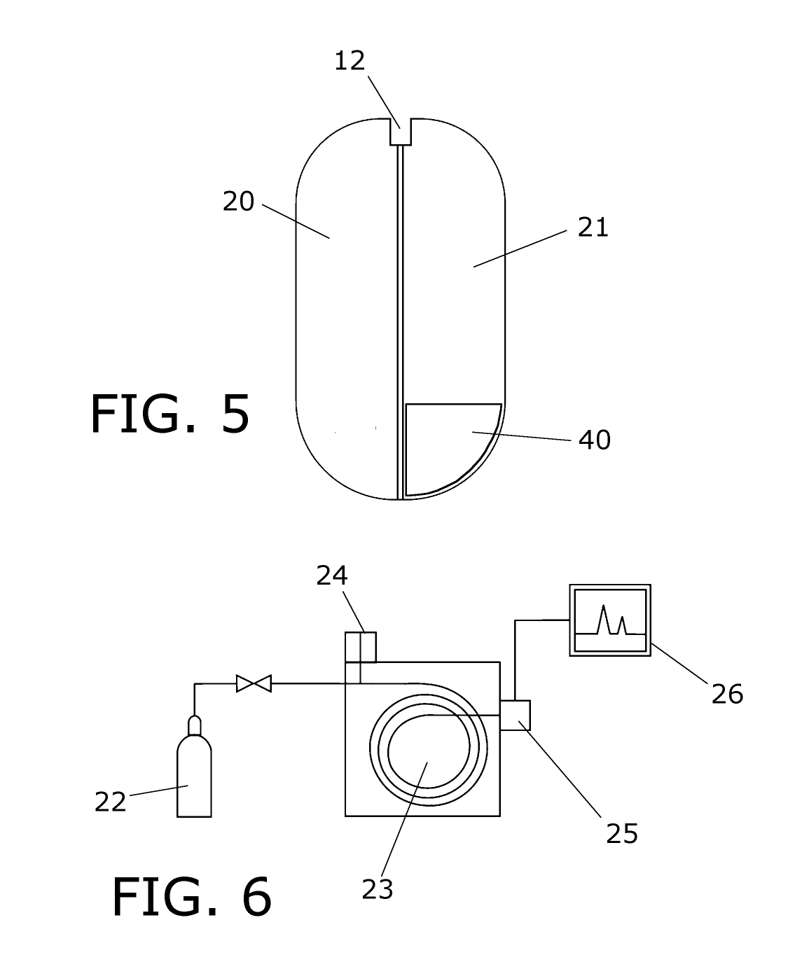

### DETAILED DESCRIPTION OF THE INVENTION

Referring now to the invention in more detail, the invention is directed to a portable spectroscopy analysis tool 10.

The invention is intended for use in testing small samples of food at the point of purchase, such as a grocery store or a restaurant. Alternate embodiments, which may be used to test other materials at other locations, are contemplated.

The first exemplary embodiment is comprised of a tool 10 which is similar in structure and function to the glucose meters used by diabetics for home blood testing, with the following modifications. Due to the broad range of herbicides, pesticides, growth hormones, carcinogens, and other materials which may be subjects of concern for the user, and the immediate need for accurate test results based on a small sample, the tool 10 preferably provides a means for testing the content of a sample 30 by employing gas chromatography-mass spectrometry (GC-MS) 30. Alternate embodiments, employing alternate methods of chromatography such as liquid chromatography (LC), are contemplated.

The tool 10 is therefore principally comprised of two analytical devices: a gas chromatograph and a mass spectrometer. Preferably, the interior of the tool 10 is divided longitudinally into a left chamber 20 and a right chamber 21. The left chamber 20 housing the carrier gas 22 which is connected to the column oven 23 of the gas chromatograph 30. The rectangular aperture providing the sample injector 24. The right chamber 21 housing the detector 25 and the mass spectrometer 26.

The tool 10 is comprised of a rectangular case with rounded corners, which provides a small, rectangular aperture 12 on its top surface and a visual display 13, an activation button 14, and an indicator light 15 on its front surface. A test strip 11, similar to those used by diabetics for blood testing, may be inserted into the aperture 12. A plurality of test strips 11, such as 20 test strips 11, is provided with the tool 10 and a larger plurality, such as 100 test strips 11, is available separately. Preferably, the tool 10 is powered by a rechargeable battery 40 and provides a Universal Service Bus (USB) port 41 on its bottom surface or left side surface. The USB port may be used to transfer test data to a different device, such as a laptop, or to recharge the battery, by inserting a removable recharging cable which is provided.

The indicator light 15 signals the operating status of the tool 10. Continuous illumination indicates that the tool 10 has been activated and is ready to conduct a test. Slow flashing indicates that the test strip 11 has been correctly inserted into the aperture 12 and that the tool 10 is conducting its analysis. Rapid flashing indicates that the analysis is complete and results are available.

Electronic components of the first exemplary embodiment preferably include a microprocessor, a memory device, a test chamber directly beneath the aperture 12, a visual display 13, an activation button 14, an indicator light 15, and a rechargeable battery.

To use the first exemplary embodiment, if the food item is a fluid substance the sample can be tested by dipping the test strip 11 into the fluid substance. If the food item is a solid or semi solid substance, then the user can dissolve a small shaving of a food sample into a solvent such as water, and dip the test strip 11 into the resulting solution. The test strip 11 is then inserted into the aperture 12 which is provided on the top surface of the tool 10, and the visual display 13 provides a chemical analysis of the sample within seconds.

The tool 10 and the test strip 11 are preferably manufactured from rigid, durable materials which are corrosion resistant, such as stainless steel, copper alloy, brass, aluminum alloy, and plastic. The visual display 13 and the indicator light 15 are preferably manufactured from a rigid, durable material which is transparent, such as plastic or malacrylate. The indicator light 15 is preferably manufactured from a flexible, durable material, such as silicone or plastic. The recharging cable is preferably manufactured from braided copper alloy wire sheathed in plastic.

Components, component sizes, and materials listed above are preferable, but artisans will recognize that alternate components and materials could be selected without altering the scope of the invention.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

I claim:

1. A portable spectroscopy analysis tool, comprising:
a. a rectangular case;
b. said rectangular case housing a gas chromatograph and a mass spectrometer;
c. said rectangular housing a sample injector accessible by a small rectangular aperture in the rectangular housing;
d. a visual display;
e. an activation button; and
f. an indicator light;
such that, a test strip containing a sample, is inserted into the sample injector through the aperture and the gas chromatograph and mass spectrometer identify the contents of the sample.
